(19) United States
(12) Patent Application Publication
(10) Pub. No.: US 2021/0035656 A1
(43) Pub. Date: Feb. 4, 2021

(54) SYSTEMS AND METHODS FOR JOINT EVENT SEGMENTATION AND BASECALLING IN SINGLE MOLECULE SEQUENCING

(71) Applicant: Seagate Technology LLC, Cupertino, CA (US)

(72) Inventors: Raman VENKATARAMANI, Longmont, CO (US); William M. RADICH, Longmont, CO (US)

(21) Appl. No.: 16/525,914

(22) Filed: Jul. 30, 2019

(51) Int. Cl. G16B 30/00

(52) U.S. Cl. CPC G16B 30/00

(57) ABSTRACT

A system comprises a joint event segmentation and basecalling module to accept raw current signals read from a DNA strand, wherein the raw current signals span one or more events each representing a single base movement in an underlying DNA base sequence. The joint event segmentation and basecalling module combines a run-length tracking hidden Markov model (HMM) for event detection and a de Bruijn HMM for basecalling in a single joint HMM, wherein the run-length HMM tracks duration of a current event in the DNA base sequence and the de Bruijn HMM tracks a local k-mer of the current event in the DNA base sequence. The joint event segmentation and basecalling module then utilizes the single joint HMM to process the raw current signals to track both the local k-mer and the duration of the current event in the DNA base sequence simultaneously via dynamic programming.

### SUMMARY

Provided herein is a system that includes a joint event segmentation and basecalling module configured to accept raw current signals sequenced from a DNA strand, wherein the raw current signals span one or more events each representing a single base movement in an underlying DNA base sequence. The joint event segmentation and basecalling module combines a run-length tracking hidden Markov model (HMM) for event detection and a de Bruijn HMM for basecalling in a single joint HMM, wherein the run-length HMM tracks duration of a current event in the DNA base sequence and the de Bruijn HMM tracks a local k-mer of the current event in the DNA base sequence via a de Bruijn graph. The joint event segmentation and basecalling module then utilizes the single joint HMM to process the raw current signals to track both the local k-mer and the duration of the current event in the DNA base sequence simultaneously via dynamic programming.

These and other features and advantages will be apparent from a reading of the following detailed description.

### BRIEF DESCRIPTION OF DRAWINGS

FIG. 1 shows an example of a HMM-based architecture for simultaneous event segmentation and basecalling according to one aspect of the present embodiments.

FIG. 2A depicts an example of the DNA strand being sequenced by the DNA sequencer and FIG. 2B depicts an example of the raw current signals of the sequenced DNA strand by the DNA sequencer spanning four events according to one aspect of the present embodiments.

FIG. 3 depicts an example of a structure of the run-length tracking HMM for event segmentation according to one aspect of the present embodiments.

FIG. 4 depicts an example of the de Bruijn graph with 2-mer states for the DNA alphabet according to one aspect of the present embodiments.

FIG. 5 depicts an example showing stay edges at the state GACG along with all its incoming and outgoing edges according to one aspect of the present embodiments.

FIG. 6 depicts an example of pseudo code of an approach for joint event detection and basecalling using dynamic programming according to one aspect of the present embodiments.

FIG. 7 depicts an example of a HMM graph illustrating states and edges for the example of U=GACG and V=ACGT according to one aspect of the present embodiments.

FIG. 8 depicts a flowchart of an example of a process to support simultaneous event segmentation and basecalling according to one aspect of the present embodiments.

### DESCRIPTION

Before various embodiments are described in greater detail, it should be understood that the embodiments are not limiting, as elements in such embodiments may vary. It should likewise be understood that a particular embodiment described and/or illustrated herein has elements which may be readily separated from the particular embodiment and optionally combined with any of several other embodiments or substituted for elements in any of several other embodiments described herein.

It should also be understood that the terminology used herein is for the purpose of describing the certain concepts, and the terminology is not intended to be limiting. Unless defined otherwise, all technical and scientific terms used herein have the same meaning as commonly understood in the art to which the embodiments pertain.

Unless indicated otherwise, ordinal numbers (e.g., first, second, third, etc.) are used to distinguish or identify different elements or steps in a group of elements or steps, and do not supply a serial or numerical limitation on the elements or steps of the embodiments thereof. For example, “first,” “second,” and “third” elements or steps need not necessarily appear in that order, and the embodiments thereof need not necessarily be limited to three elements or steps. It should also be understood that the singular forms of “a,” “an,” and “the” include plural references unless the context clearly dictates otherwise.

Some portions of the detailed descriptions that follow are presented in terms of procedures, methods, flows, logic blocks, processing, and other symbolic representations of operations performed on a computing device or a server. These descriptions and representations are the means used by those skilled in the data processing arts to most effectively convey the substance of their work to others skilled in the art. In the present application, a procedure, logic block, process, or the like, is conceived to be a self-consistent sequence of operations or steps or instructions leading to a desired result. The operations or steps are those utilizing physical manipulations of physical quantities. Usually, although not necessarily, these quantities take the form of electrical or magnetic signals capable of being stored, transferred, combined, compared, and otherwise manipulated in a computer system or computing device or a processor. It has proven convenient at times, principally for reasons of common usage, to refer to these signals as transactions, bits, values, elements, symbols, characters, samples, pixels, or the like.

It should be borne in mind, however, that all of these and similar terms are to be associated with the appropriate physical quantities and are merely convenient labels applied to these quantities. Unless specifically stated otherwise as apparent from the following discussions, it is appreciated that throughout the present disclosure, discussions utilizing terms such as “storing,” “determining,” “sending,” “receiving,” “generating,” “creating,” “fetching,” “transmitting,” “facilitating,” “providing,” “forming,” “detecting,” “decrypting,” “encrypting,” “processing,” “updating,” “instantiating,” or the like, refer to actions and processes of a computer system or similar electronic computing device or processor. The computer system or similar electronic computing device manipulates and transforms data represented as physical (electronic) quantities within the computer system memories, registers or other such information storage, transmission or display devices.

It is appreciated that present systems and methods can be implemented in a variety of architectures and configurations. For example, present systems and methods can be implemented as part of a distributed computing environment, a cloud computing environment, a client server environment, hard drive, etc. Embodiments described herein may be discussed in the general context of computer-executable instructions residing on some form of computer-readable storage medium, such as program modules, executed by one or more computers, computing devices, or other devices. By way of example, and not limitation, computer-readable storage media may comprise computer storage media and communication media. Generally, program modules include routines, programs, objects, components, data structures, etc., that perform particular tasks or implement particular data types. The functionality of the program modules may be combined or distributed as desired in various embodiments.

Computer storage media/drive can include volatile and nonvolatile, removable and non-removable media implemented in any method or technology for storage of information such as computer-readable instructions, data structures, program modules, or other data. Computer storage media can include, but is not limited to, random access memory (RAM), read only memory (ROM), electrically erasable programmable ROM (EEPROM), flash memory, or other memory technology, compact disk ROM (CD-ROM), digital versatile disks (DVDs) or other optical storage, magnetic cassettes, magnetic tape, magnetic disk storage or other magnetic storage devices, or any other medium that can be used to store the desired information and that can be accessed to retrieve that information.

DNA sequencing has undergone several phases of innovation starting from the classic sequencing methods of Sanger dideoxy (terminator base) method and Maxam-Gilbert (chemical cleavage) method. The Sanger dideoxy method works by replicating a strand but stopping at a random instance of the specific base, while the Maxam-Gilbert method works by selectively cleaving the DNA strand at instances of a specific chosen base (A, C, G or T). Both methods are similar in that they generate short DNA fragments (of random lengths) terminating at an instance of the selected base. A technique called polyacrylamide gel electrophoresis is used to measure the lengths of the resulting fragments, which determines the positions of the specific base in the original DNA strand. The process is done separately for each of the four bases to get the complete DNA sequence. A limitation of these methods is that they only work well for sequences that are 100 bases long. Beyond this limit the uncertainty in the position of each base becomes unacceptable. As a result, longer sequences have to be broken down, sequenced individually, and stitched together like pieces of a jig saw puzzle using genome assembly algorithms.

The second generation of DNA sequencing saw the rise of massively parallel sequencers that were still limited to processing short fragments. The idea of “single molecule sequencing” is to avoid fragmentation of the DNA and try to sequence the entire single stranded DNA (ssDNA) molecule in a single pass. Currently, single-molecule sequencers from Pacific Biosciences (PacBio) and Oxford Nanopore Technologies (ONT) can sequence molecules over 10 kilobases long. PacBio's single-molecule real-time sequencer (SMRT) uses an optical waveguide system to sequence DNA, while ONT uses a nanopore device with a sensor to measure electrical currents.

Nanopore DNA sequencing is a single molecule sequencing technology that promises low-cost high-throughput DNA sequencing for medical applications as well as DNA based data storage. A nanopore DNA sequencing device contains a tiny pore through which a negatively charged single strand of DNA from the sample is made to pass through a nanopore channel under an external electric field by a process called electrophoresis. The width of the nanopore channel is in the order of, e.g., 2 nm, just wide enough for a single stranded DNA molecule (ssDNA) to pass through. The raw signal measured by the device sensor is a sampled transverse ionic or tunneling current between two electrodes, wherein the current depends on the base sequence in the DNA strand. Each base produces a characteristic current response. By measuring the changes in the measured current with the passing of each base in the DNA sequence through the pore, the method may detect the bases (nucleotides) in the ssDNA sequence.

The main computational problem associated with nanopore DNA sequencing is using the raw current signal to recover the underlying DNA base sequence, known as the basecalling problem. However, it is often simpler to first identify events boundaries (event detection problem) via segmentation and post process the identified events to recover the base sequence via basecalling. During such two-step approach, the sampled current signal is first segmented into events, wherein each event corresponds to individual bases transiting through the nanopore channel. Next, the event information is fed to a basecaller to detect the base sequence in the DNA strand. The basecaller is typically implemented using a hidden Markov model (HMM), which is a statistical Markov model in which the system being modeled is assumed to be a Markov process with unobservable (i.e. hidden) states, or neural-networks.

The two-step process of event segmentation followed by post-segmentation basecalling is not necessarily optimal because the event segmentation step does not use the interdependency in the signal levels caused by inter-symbol interference (ISI). Furthermore, such two-step process typically needs to over-segment the raw signal to keep the basecaller complexity under control and it may not be able to handle cases where the raw data is under-segmented beyond the HMM capability.

A new approach is proposed that contemplates systems and methods to support a new architecture to perform joint event segmentation and basecalling from a sampled current signal from a DNA sequencer via a hidden Markov model (HMM). Here, the HMM is based on a dynamic programming algorithm that tracks both the duration of the current event (event run-length) and the local k-mer pattern in the DNA strand/base sequence. Compared to the two-step process discussed above, the proposed approach utilizes a joint HMM to operate directly on the raw signal samples sensed by single bio-molecule sequencers in general, including nanopore DNA sequencing. Since the proposed approach is derived from first principles/terms using Bayesian estimation, it is provably optimal for the class of models considered. In addition, the joint event segmentation and basecaller is aware of the underlying structure (base sequence) that causes the signal levels to be interdependent. The proposed approach provides an efficient way to compute the branch metrics for the HMM with O(1) complexity per state and the overall complexity of the HMM is O(4^{M}K) where M is the memory size of the ISI in the base response and K is the maximum event duration tracked by the HMM.

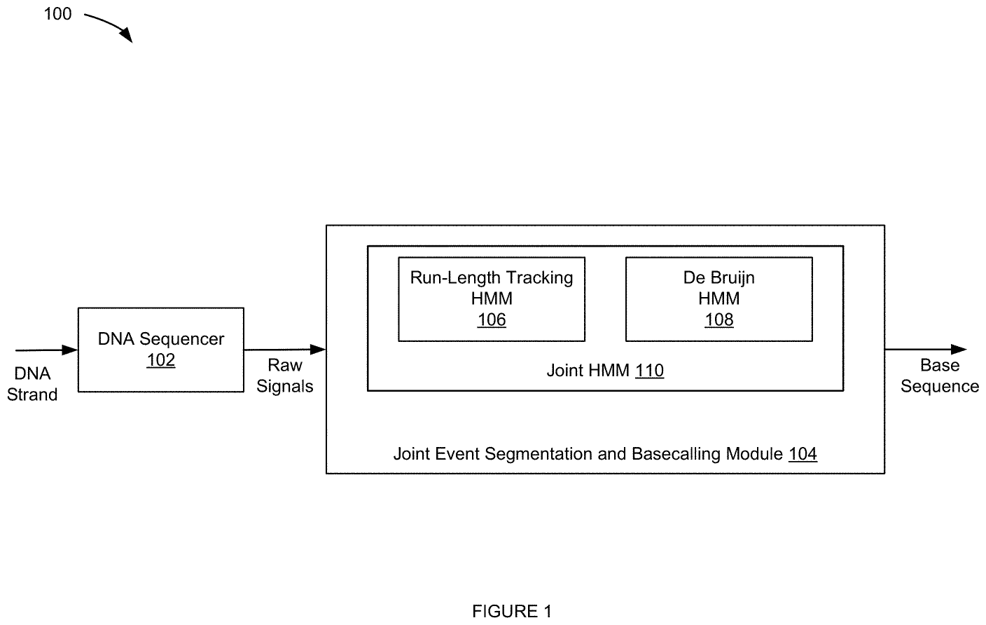

Referring now to FIG. 1, an example of a novel HMM-based architecture 100 for simultaneous event segmentation and basecalling according to one aspect of the present embodiments is shown. The architecture 100 includes at least a DNA sequencer 102 and a joint event detection and basecalling module 104. Each component of the architecture 100 runs on a host (not shown), which includes one or more processors with software instructions stored in a storage unit such as a non-volatile memory (also referred to as secondary memory) of the host for practicing one or more processes. When the software instructions are executed by the one or more processors of the host, at least a subset of the software instructions is loaded into a memory unit (also referred to as primary memory) by the host, which becomes a special purposed one for practicing the processes. The processes may also be at least partially embodied in the host into which computer program code is loaded and/or executed, such that, the host becomes a special purpose computing unit for practicing the processes. When implemented on a general-purpose computing unit, the computer program code segments configure the computing unit to create specific logic circuits. In some embodiments, each host can be a computing device, a communication device, a storage device, or any computing device capable of running a software component. Each host has a communication interface (not shown), which enables the engines to communicate with each other, the user, and other devices over one or more communication networks following certain communication protocols, such as TCP/IP, http, https, ftp, and sftp protocols.

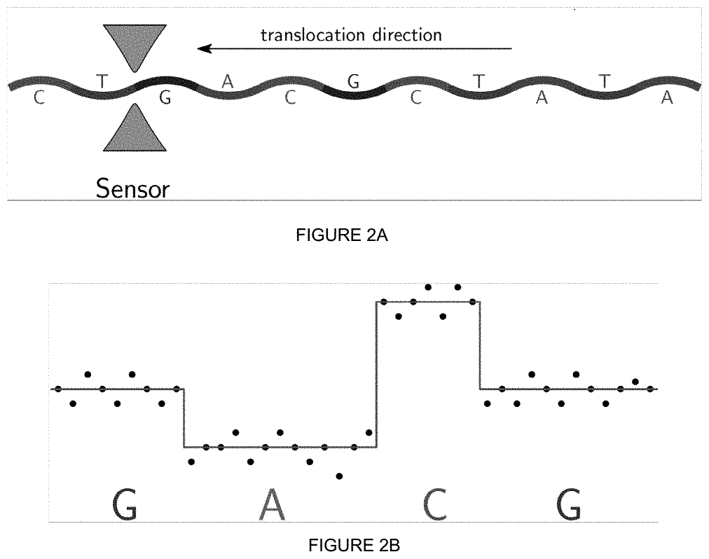

In the example of FIG. 1, the DNA sequencer 102 is configured to accept and sequence a DNA strand into raw current signals. Here, the DNA sequencer 102 can be but is not limited to a nanopore DNA sequencer. FIG. 2A depicts an example of the DNA strand being sequenced by sensors of the DNA sequencer 102 in the translocation direction and FIG. 2B depicts an example of the raw current signals of the sequenced DNA strand by the DNA sequencer 102 spanning four events, wherein the dots represent sampled current samples and the line represents the ideal level corresponding to each event representing a single base movement in the DNA strand through the nanopore DNA sequencer 102.

In the example of FIG. 1, the joint event segmentation and basecalling module 104 takes the raw current signals of the sequenced DNA strand as its input. The joint event segmentation and basecalling module 104 is then configured to combine a run-length tracking HMM (or RL-HMM) 106 for event detection and a de Bruijn HMM 108 for basecalling in a way that the states of a single joint HMM 110 tracks both a local k-mer and a duration of a current event in a DNA base sequence simultaneously. Here, the local k-mer is a subsequence of length k contained within DNA base sequence. In some embodiments, the joint event segmentation and basecalling module 104 is configured to utilize the underlying structure of the DNA base sequence, wherein the signal levels for one event and the next event are interdependent due to the shared part of the underlying DNA base sequence.

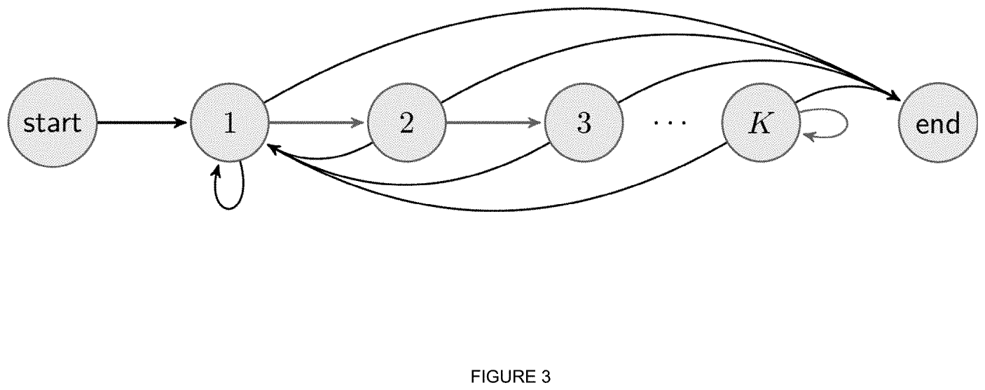

In some embodiments, the run-length tracking HMM 106 is configured to encode the run-length or duration of the current event, rather than signal level of the current event, into the HMM state. In some embodiments, the run-length tracking HMM 106 is configured to estimate the event levels on-the-fly at each HMM state based on the samples in the current event at that state. Since the signal level is not encoded at all, encoding the run-length or duration of the current event works accurately even for large or even continuous level sets without level quantization. FIG. 3 depicts an example of a structure of the run-length tracking HMM 106 for event segmentation. As shown by the example of FIG. 3, the states are labeled with state index m=1, 2, . . . , K, wherein the state index m represents the run length of current event, i.e., the m most recent samples make up the current event. The outgoing edges from state m are to state m+1, representing the extension of the current run, and to state 1, representing the end of the current event. In some embodiments, the branch metrics are constructed in terms of either a maximum likelihood (ML) path, which is path with the least accumulated path metric through the HMM, or a Bayesian scoring function. In some embodiments, Viterbi algorithm is used to compute the HMM state sequence with the least accumulated path metric, which immediately provides information on how to segment the observation into events.

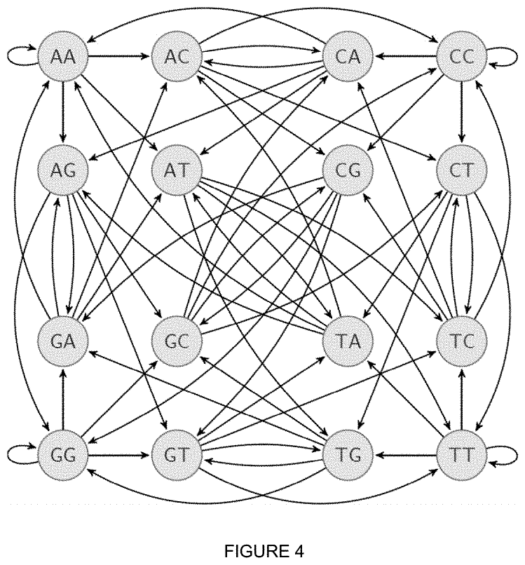

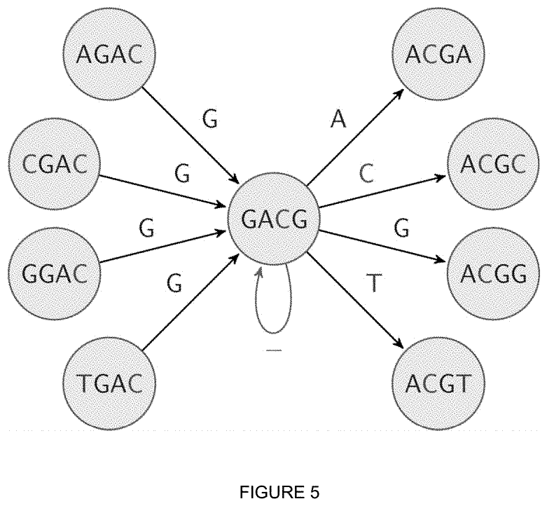

In some embodiments, the de Bruijn HMM 108 used for basecalling is based on the de Bruijn graph, which is a graph whose states are k-mer substrings and had edges (U, V) if the length-(k−1) suffix of state U equals the length-(k−1) prefix of state V. For post-segmentation basecalling, the de Bruijn HMM 108 is configured to minimize under-segmentation where two or more true events are detected as a single merged event even if one or more individual events may be detected as multiple smaller events as a result. FIG. 4 depicts an example of the de Bruijn graph with 2-mer states for the DNA alphabet. In some embodiments, “stay edges,” which are self loops from each state to itself, are included in the de Bruijn graph to handle over-segmentation. FIG. 5 depicts an example showing stay edges at the state GACG along with all its incoming and outgoing edges. At state GACG there are incoming edges from XGAC and outgoing edges to ACGX where “X” represents each of the four base choices. The label on each edge is the new base appended to the destination state. In some embodiments, the branch metrics are modeled based on the probabilistic models for the event information which in turn are learned from training data.

In some embodiments, the joint event segmentation and basecalling module 104 is configured to adopt a simple model for the raw current signals for a typical nanopore DNA sequence wherein each raw current signal is modeled as a (noisy) piecewise-constant over each event where each event represents the movement of a single base through the nanopore channel. In the discussions below, the following definitions and notation are adopted. Let B={A, C, G, T} denote the DNA base (nucleotide) alphabet and b={bₖ∈B} a base sequence in the DNA strand being read by the sequencer. Let d={dₖ} and λ={λₖ} be shorthand for the sequence of durations and ideal (noise-free) levels of all events that make up the observation sequence. Let Eₖ denote the temporal support of the k-th event:

$${E}_{k} = \{ j \text{:} { \sum }_{i < k} {d}_{i} < j \leq {\sum }_{i \leq k} {d}_{i} \} \qquad ( 1 )$$

Since the event duration dₖ may be very weakly correlated with from one event to the next, in some embodiments, the event durations dₖ can be modeled as independent and identically distributed (IID) and independent of the level variable kₖ with known a priori probability distribution P (dₖ).

In some embodiments, the joint event segmentation and basecalling module 104 is configured to adopt a signal model for noisy raw samples in the k-th event as

sₙ=λₖ+wₙ (2)

where wₙ˜ [symbol] (0,σₖ²) for n∈Eₖ is additive IID Gaussian noise with zero mean variance σₖ² and kₖ is the level of the k-th event that may depend on the local k-mer pattern. In some embodiments, the joint event segmentation and basecalling module 104 is configured to adopt a slightly more complex noise model by introducing correlations between noise samples with an event:

w(Eₖ)˜ [symbol] (0;Σₖ) (3)

where w(Eₖ)={wₙ: n∈Eₖ} is shorthand for the vector of noise samples in the k-th event, and Σₖ is a dₖ×dₖ covariance matrix. One way to introduce correlations is by using a common noise source for each event. Specifically, we model wₙ as a sum of two IID noise components:

wₙ=vₙ+uₖ;n∈Eₖ

with vₙ˜ [symbol] (0,σₖ²) and uₖ˜ [symbol] (0,τₖ²) (indexed by k rather than n so that it affects all noise samples in the k-th event). Here, vₙ represents the sensor measurement noise with a bandwidth high enough to be modeled as white noise. And uₖ represents a slowly varying noise that includes unexplained noise sources such as interference from other bio-molecules or residual ISI not modeled by the signal. This results in a dₖ×dₖ covariance matrix with the following two-parameter form:

$${Σ}_{k} = {σ}_{k}^{2} I + {τ}_{k}^{2} e {e}^{T} = ( \begin{matrix}{τ}_{k}^{2} + {σ}_{k}^{2} & {τ}_{k}^{2} & … & {τ}_{k}^{2} \\ {τ}_{k}^{2} & {τ}_{k}^{2} + {σ}_{k}^{2} & & ⋮ \\ ⋮ & & ⋱ & {τ}_{k}^{2} \\ {τ}_{k}^{2} & … & {τ}_{k}^{2} & {τ}_{k}^{2} + {σ}_{k}^{2}\end{matrix} )$$

where e=(1, 1, . . . , 1)^{T} is the vector with all elements being ones. This model generalizes the simpler IID noise model when we set τₖ²=0. All these noise parameters can also be modeled as look-up tables Φ_{σ}[⋅]. and Φ_{τ}[⋅] applied to the local (2L+1)-mer base pattern:

σₖ²=Φ_{σ}[b_{k-L}^{k+L}],τₖ²=Φ_{τ}[b_{k-L}^{k+L}]

Although our primary focus above was a signal model for nanopore DNA sequencing, the general models for other bio-molecule sequencers are similar. All of our following methods are broadly applicable to these sequencing technologies as well.

Starting from the signal model (2) and the noise model (3), the joint event segmentation and basecalling module 104 is configured to calculate log-probability of the observed raw signal samples as

$$log P ( s \vert d , b ) = {\sum }_{k} log P ( s ( {E}_{k} ) \vert {d}_{k} , {b}_{k - L}^{k + L} ) \qquad ( 4 )$$

wherein b={bₖ} and d={dₖ} denote the base sequence and event durations, respectively and s(Eₖ) denote the raw signal samples in event Eₖ defined in (1). All of λₖ,σₖ and τₖ² are all functions of the local M-mer pattern b_{k-L}^{k+L} with M=2L+1. By assumption, the durations {dₖ} are independent and identically distributed (IID) with a known priori probability distribution function (PDF) P(dₖ). Therefore,

$$P ( d ) = {\prod }_{k} P ( {d}_{k} ) \qquad ( 5 )$$

If there is no prior information about the base sequence, the joint event segmentation and basecalling module 104 is configured to use Bayesian estimation to find the durations d and base sequence b as follows:

$$( \hat{d} , \hat{b} ) = arg {max}_{d , b} log P ( s , d , b ) = arg {max}_{d , b} P ( s \vert d , b ) P ( d )$$

Taking the logarithm of the above equation and applying (4) and (5), (\hat{d}, \hat{b}) can be calculated as:

$$( \hat{d} , \hat{b} ) = arg {max}_{d , b} {\sum }_{k} [ log P ( s ( {E}_{k} ) \vert {d}_{k} , {b}_{k - L}^{k + L} ) + log P ( {d}_{k} ) ]$$

If there is prior information on the base sequence in the form of a Markov model P(b_{k+L}|b_{n-L}^{k+L-1}), the joint event segmentation and basecalling module 104 is configured to incorporate a log-probability of this prior information into the above expression. Using (2), (3) and the standard expression for a multi-dimensional Gaussian PDF, the first term in the above summation can be calculated as:

$$log P ( s ( {E}_{k} ) \vert {d}_{k} , {b}_{k - L}^{k + L} ) = - \frac{1}{2} {( s ( {E}_{n} ) - {λ}_{k} e )}^{T} {\sum }_{k}^{- 1} ( s ( {E}_{n} ) - {λ}_{k} e ) - \frac{1}{2} log ( {( 2 π )}^{{d}_{k}} det {\sum }_{k} ) \qquad ( 7 )$$

wherein detΣₖ can be calculated as

$$\begin{matrix}{det Σ}_{k} = ( {σ}_{k}^{2} + {d}_{k} {τ}_{k}^{2} ) {σ}_{k}^{2 ( {d}_{k} - 1 )} & ( 8 ) \\ {Σ}_{k}^{- 1} = \frac{I}{{σ}_{k}^{2}} - \frac{{τ}_{k}^{2} e {e}^{T}}{{σ}_{k}^{2} ( {σ}_{k}^{2} + {d}_{k} {τ}_{k}^{2} )} & ( 9 )\end{matrix}$$

Using (9), the first term in (7) can be reduced to the following simple form:

$$- \frac{1}{2 {σ}_{k}^{2}} {\sum }_{n ∈ {E}_{k}} {( {s}_{n} - {λ}_{k} )}^{2} + \frac{{τ}_{k}^{2}}{2 {σ}_{k}^{2} ( {σ}_{k}^{2} + {d}_{k} {τ}_{k}^{2} )} {( {\sum }_{n ∈ {E}_{k}} ( {s}_{n} - {λ}_{k} ) )}^{2} \qquad ( 10 )$$

while the second term in (7), being independent of the observations, can be precomputed using (8) and stored in a look-up table for efficiency by the joint event detection and basecalling module 104.

In some embodiments, the joint event segmentation and basecalling module 104 is configured to solve the equation (6) using dynamic programming (DP). The idea of dynamic programming is to reduce the main problem to a simple post-processing step in terms of some or all smaller sub-problems. These sub-problems then are solved sequentially going from smallest to largest in size. Let Vₖ=b_{k-L+1}^{k+L} denote the (M−1)-mer pattern referred to as the “k-mer state” at time k. The state sequence V={Vₖ} uniquely describes the base sequence b={bₖ} and vice versa. Furthermore, the k-mer b_{k-L}^{k+L} can be compactly represented by the pair (Vₖ₋₁; Vₖ), which also represents an edge in the de Bruijn graph g whose states are (M−1)-mers. As such, the Bayesian estimation problem (6) can be calculated as a maximization problem/function using an additive scoring model, wherein the goal is to maximize a sum of individual event scores as follows:

$$( \hat{d} , \hat{V} ) = arg {max}_{d , v} {\sum }_{k} S ( {V}_{k - 1} , {V}_{k} , {E}_{k} ) \qquad ( 11 )$$

with the score for the k-th event defined as

S(Vₖ₋₁,Vₖ,Eₖ)) [symbol] log P(s(Eₖ)|dₖ,b_{k-L}^{k+L}+log P(dₖ) (12)

For dynamic programming, let F [n, V], for n≤N and each ending at state V. Denote the best score for sub-problem with partial observation sequence S₁ⁿ and let E_{n,mi}={j:n−m+1≤j≤n} denote the event of length m ending at index n. Then, starting with F [0, V]=0, F [n, V] can be updated recursively as follows:

$$F [ n , V ] = {max}_{{1 \leq m \leq n}_{U ∈ p ( V )}} \{ F [ n - m , U ] + S ( U , V , {E}_{n} , m ) \}$$

where p(V)={U:(U,V) is an edge in [symbol] } is the set of parent states of V.

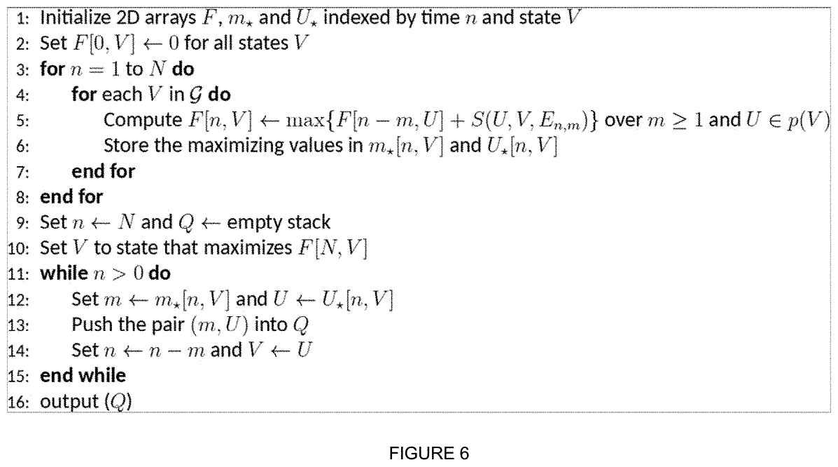

In the maximization F[n, V] above, m represents the duration of the last event and U is the ending state for the penultimate event for the sub-problem of size n, wherein U must be such that (U, V) is an edge in the de Bruijn graph [symbol] . In some embodiments, the joint event segmentation and basecalling module 104 is configured to calculate the maximization function sequentially over n for each V∈ [symbol] . For each n and V, the joint event segmentation and basecalling module 104 is configured to store the optimal values form and U (trace-back information) in memory. At the end, the joint event segmentation and basecalling module 104 is configured to trace back to recover the event durations and sequence of k-mer states (and hence base movements). FIG. 6 depicts an example of pseudo code of an approach for joint event detection and basecalling using dynamic programming as discussed above. As shown in FIG. 6, the output is a first-in last-out (FILO) stack Q containing pairs (m, U) interpreted as the event duration and k-mer state. Since these pairs are pushed into Q in reverse order, they would be popped out in the correct order.

In some embodiments, the joint event segmentation and basecalling module 104 is configured to reformulate the dynamic programming algorithm as running the Viterbi algorithm on the following joint HMM 110 that combines the run-length HMM 106 and the de Bruijn HMM 108 to track both the local k-mer state and the event duration (run-length). In some embodiments, the HMM states are labeled by a pair (U, m) where U is a k-mer and m is the duration of the current event. The state (U, m) has an outgoing edge to (U, m+1) to represent the extension of the current event. It also has edges to states (V, 1) to represent the transition to a new event for all V such that (U, V) is an edge in the de Bruijn graph [symbol] . In some embodiments, where m is large, the joint event segmentation and basecalling module 104 is configured to limit the HMM complexity by picking a sufficiently large parameter K and merging all states with m≥K into a single state labeled m=K For a slightly technical reason we also need to include the k-mer V along with U in the terminal states with m=K. This will be clear shortly when we describe the branch metrics below. Such HMM implementation with a limit on the maximum run-length effectively achieves linear complexity and real-time segmentation and basecalling. In summary, the main HMM states are labeled (U, m) with U∈ [symbol] and 1≤m≤K−1. Additional HMM states are of the form (U, V, K) for all edges (U, V) in [symbol] , wherein the following is a complete list of all the edges in the HMM 110:

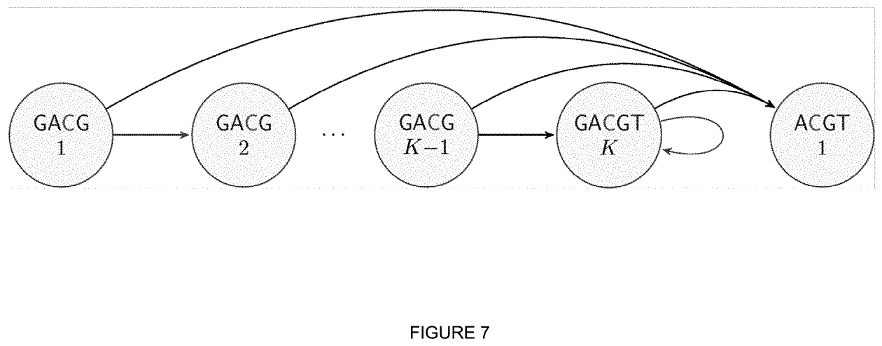

1. (U,m) has edges to (U,m+1) for 1≤m≤K−2 and (V,1)
2. (U,K−1) has edges to (U,V,K) and (V,1)
3. (U,V,K) has edges to (U,V,K) and (V,1)
where U and V are any k-mers in [symbol] such that (U, V) is an edge in [symbol] . FIG. 7 depicts an example of a HMM graph illustrating the above states and edges for the example U=GACG and V=ACGT. The full HMM 110 is obtained by allowing U and V to take on all values (k-mers) such that (U, V) is an edge in [symbol] .

In some embodiments, the joint event segmentation and basecalling module 104 is configured to translate the DP into the language and form of HMMs as follows. Specifically, the branch metrics in the HMI 110, expressed in terms of the scoring function (12) discussed above, are defined for event extension edges and are set to zero:

βₙ[(U,m)→(U,m+1)]=0 for 1≤m≤K−2;

βₙ[(U;K−1)→(U,V,K)]=0

because a simple approach is adopted in the way the signal samples are processed. In some embodiments, the (accumulated) metric of involving these signal samples are computed by means of the event scoring function, only when the joint event segmentation and basecalling module 104 commits to ending the current event, i.e., on event transition edges. The branch metric for these edges are

βₙ[(U,m)→(V,1)]=−S(U,V,E_{n-1,m}) for 1≤m≤K−1;

βₙ[(U,V,K)→(V,1)]=−S(U,V,E_{n-1,K})

Finally, the branch metric for the self-loop at state (U,V,K),

βₙ[(U,V,K)→(U,V,K)]=−S(U,V,E_{n,K+1})+S(U,V,E_{n,K}),

is carefully chosen to guarantee the correct path metric if the event duration were exactly K+1. As such, the HMM 110 computes path metrics correctly for all event durations up to length K+1. Beyond that it is an approximation, but generally a good one, if K is sufficiently large. In some cases K only needs to be long enough such that P(dₖ) has a geometric tail distribution for d≥K to get correct results. One such example is when the noise has a simple IID noise model, i.e. when τₖ²=0. Note that the self-loop metric depends on both U and V: this is the reason the terminal states is labeled to include both U and V. Additionally, it can be shown that, ignoring the finiteness of K, that the score F[n, V] in the DP equals negative of the state metric at state (V,1) at time n+1. In some embodiments, the Viterbi algorithm is used to compute the state sequence with the least accumulated path metric in the HMM.

Since the base alphabet [symbol] has 4 elements, the number of (M−1)-mers is 4^{M-1}. A simple counting argument shows that there are 4^{M-1}(K−1)+4^{M-1}(K+3)=states in the HMM 110. Similarly, there are 4^{M} (K+1) edges with nonzero branch metrics. The bulk of the computation needed for the branch metric βₙ[(U,m)→(V,1)] is the expression (10) as it involves many signal samples. However, that computation can be done efficiently in terms of cumulative sums of (sₙ₋ₘ−λₖ) and (sₙ₋ₘ−λₖ)² over m from 1 to K. This results in a complexity of only O(1) per edge. The overall complexity of the HMM 110 per time sample is thus O(4^{M}K).

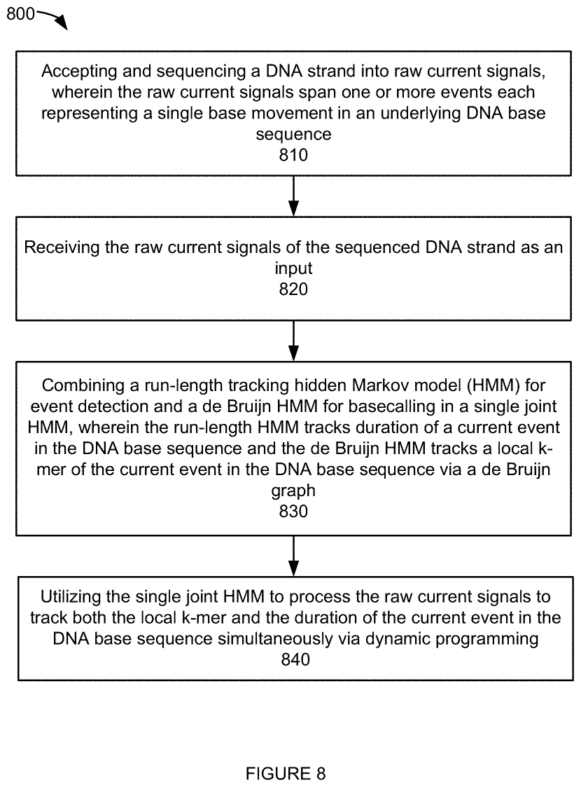

FIG. 8 depicts a flowchart of an example of a process to support simultaneous event segmentation and basecalling. One skilled in the relevant art will appreciate that the various steps portrayed in this figure could be rearranged, combined and/or adapted in various ways. In the example of FIG. 8, a DNA strand is accepted and sequenced into raw current signals at step 810, wherein the raw current signals span one or more events each representing a single base movement in an underlying DNA base sequence. The raw current signals of the sequenced DNA strand is received as an input at step 820. A run-length tracking hidden Markov model (HMM) for event detection and a de Bruijn HMM for basecalling is combined into a single joint HMM at step 830, wherein the run-length HMM tracks duration of a current event in the DNA base sequence and the de Bruijn HMM tracks a local k-mer of the current event in the DNA base sequence via a de Bruijn graph. The single joint HMM is then utilized to process the raw current signals to track both the local k-mer and the duration of the current event in the DNA base sequence simultaneously via dynamic programming at step 840.

While the embodiments have been described and/or illustrated by means of particular examples, and while these embodiments and/or examples have been described in considerable detail, it is not the intention of the Applicants to restrict or in any way limit the scope of the embodiments to such detail. Additional adaptations and/or modifications of the embodiments may readily appear, and, in its broader aspects, the embodiments may encompass these adaptations and/or modifications. Accordingly, departures may be made from the foregoing embodiments and/or examples without departing from the scope of the concepts described herein. The implementations described above and other implementations are within the scope of the following claims.

What is claimed is:

1. A system comprising:
a DNA sequencer configured to accept and read a DNA strand into raw current signals, wherein the raw current signals span one or more events each representing a single base movement in an underlying DNA base sequence; and
a joint event segmentation and basecalling module configured to
receive the raw current signals of the sequenced DNA strand as its input;
combine a run-length tracking hidden Markov model (HMM) for event detection and a de Bruijn HMM for basecalling in a single joint HMM, wherein the run-length HMM tracks duration of a current event in the DNA base sequence and the de Bruijn HMM tracks a local k-mer of the current event in the DNA base sequence via a de Bruijn graph; and
utilize the single joint HMM to process the raw current signals to track both the local k-mer and the duration of the current event in the DNA base sequence simultaneously via dynamic programming.

2. The system as described in claim 1, wherein:
the DNA sequencer is a single molecule nanopore DNA sequencer.

3. The system as described in claim 1, wherein:
the joint event segmentation and basecalling module is configured to utilize the underlying structure of the DNA base sequence wherein signal levels for one event and the next event are interdependent due to the shared part of the DNA base sequence.

4. The system as described in claim 1, wherein:
the run-length tracking HMM is configured to encode the duration of the current event, rather than signal level of the current event, into an HMM state.

5. The system as described in claim 1, wherein:
the joint event segmentation and basecalling module is configured to model the raw current signals as one or more noisy piecewise-constants over each event where each event represents the single base movement.

6. The system as described in claim 1, wherein:
the joint event segmentation and basecalling module is configured to
adopt a noise model with correlations between observed noise samples of the raw current signals with an event; and
calculate log-probability of the observed noise samples.

7. The system as described in claim 6, wherein:
the joint event segmentation and basecalling module is configured to calculate the log-probability metric using Bayesian estimation and performing the maximization of a scoring function using dynamic programming.

8. The system as described in claim 7, wherein:
the joint event segmentation and basecalling module is configured to
calculate the scoring function sequentially for each node in the de Bruijn graph; and
trace back to recover the event durations and sequence of local k-mer states and the base movement.

9. The system as described in claim 1, wherein:
the joint event segmentation and basecalling module is configured to reformulate the dynamic programming as running the Viterbi algorithm on the joint HMM to track both the local k-mer and the duration of the current event.

10. The system as described in claim 1, wherein:
the joint event segmentation and basecalling module is configured to translate the dynamic programming into the Viterbi algorithm by defining branch metrics in the joint HMM expressed in terms of scoring functions.

11. A system comprising:
a joint event segmentation and basecalling module configured to
accept raw current signals read from a DNA strand as its input, wherein the raw current signals span one or more events each representing a single base movement in an underlying DNA base sequence;
combine a run-length tracking hidden Markov model (HMM) for event detection and a de Bruijn HMM for basecalling in a single joint HMM, wherein the run-length HMM tracks duration of a current event in the DNA base sequence and the de Bruijn HMM tracks a local k-mer of the current event in the DNA base sequence via a de Bruijn graph, wherein the local k-mer is a subsequence of length k contained within the DNA base sequence; and
utilize the single joint HMM to process the raw current signals to track both the local k-mer and the duration of the current event in the DNA base sequence simultaneously via dynamic programming.

12. The system as described in claim 11, wherein:
the joint event segmentation and basecalling module is configured to utilize the underlying structure of the DNA base sequence wherein signal levels for one event and the next event are interdependent due to the shared part of the DNA base sequence.

13. The system as described in claim 11, wherein:
the run-length tracking HMM is configured to encode the duration of the current event, rather than signal level of the current event, into an HMM state.

14. A method comprising:
accepting and reading a DNA strand into raw current signals, wherein the raw current signals span one or more events each representing a single base movement in an underlying DNA base sequence;
receiving the raw current signals of the sequenced DNA strand as an input;
combining a run-length tracking hidden Markov model (HMM) for event detection and a de Bruijn HMM for basecalling in a single joint HMM, wherein the run-length HMM tracks duration of a current event in the DNA base sequence and the de Bruijn HMM tracks a local k-mer of the current event in the DNA base sequence via a de Bruijn graph; and
utilizing the single joint HMM to process the raw current signals to track both the local k-mer and the duration of the current event in the DNA base sequence simultaneously via dynamic programming.

15. The method as described in claim 14 further comprising:
utilizing the underlying structure of the DNA base sequence wherein signal levels for one event and the next event are interdependent due to the shared part of the DNA base sequence.

16. The method as described in claim 14 further comprising:
encoding the duration of the current event, rather than signal level of the current event, into an HMM state.

17. The method as described in claim 14 further comprising:
modelling the raw current signals as one or more noisy piecewise-constants over each event where each event represents the single base movement.

18. The method as described in claim 14 further comprising:
adopting a noise model with correlations between observed noise samples of the raw current signals with an event; and
calculating log-probability of the observed noise samples.

19. The method as described in claim 18 further comprising:
calculating the log-probability metric using Bayesian estimation and performing the maximization of a scoring function using dynamic programming.

20. The method as described in claim 19 further comprising:
calculating the scoring function sequentially for each node in the de Bruijn graph; and
tracing back to recover the event durations and sequence of local k-mer states and the base movement.
